(19) United States
(12) Patent Application Publication
(10) Pub. No.: US 2022/0238200 A1
(43) Pub. Date: Jul. 28, 2022

(54) SYSTEMS AND METHODS FOR FACILITATING SELF-ADMINISTRATION OF PILLS

(71) Applicant: Vive Health LLC, Naples, FL (US)

(72) Inventor: Anthony Henry Barbier, Bonita Springs, FL (US)

(21) Appl. No.: 17/156,201

(22) Filed: Jan. 22, 2021

(51) Int. Cl. G16H 20/13; G06K 7/14; A61J 7/04

(52) U.S. Cl. CPC G16H 20/13; A61J 7/0481; A61J 7/0436; G06K 7/1417

(57) ABSTRACT

A pill storage device includes a pill storage casing. The pill storage device casing defines a compartment, and the compartment includes a compartment interior. The compartment interior includes a visual token. The visual token is detectable by a scanning sensor to indicate that contents have been removed from the compartment interior.

### BACKGROUND

Many individuals experience conditions that are treatable and/or manageable via medication. In some instances, medication for addressing such conditions is available in a format that allows users to self-administer the medication without the supervision of medical practitioners. For example, many medications are available in a pill format, allowing users to easily ingest a desired amount of medication (e.g., a predetermined number of pills) at desired time intervals. By providing medication in the form of pills, users may manage medical conditions without regular supervision and can advantageously experience increased user autonomy, self-efficacy, dignity, etc.

However, in view of the lack of supervision associated with self-administration of medicinal pills, many individuals inadvertently fail to take their medicinal pills according to a prescribed or desired schedule. For instance, users may fail to take their medication at appropriate and/or regular time intervals (e.g., appropriately offset from other instances of pill ingestion) or may entirely miss dose administration instances. Indeed, many users fail to complete pill administration regimens due to neglect. Failure of users to self-ingest medicinal pills can lead to decreased efficacy of medication, prolonged treatment periods, and/or adverse patient outcomes.

Furthermore, pills that do not include medication (e.g., drug-free pills) are also available and widely used to allow users to regularly ingest desired ingredients and/or supplements to improve, maintain, and/or promote personal health and/or wellbeing. However, many users who desire to regularly ingest such pills forget to do so in accordance with their desires, causing many users to fail to attain the benefits expected to be provided by such pills.

For at least the foregoing reasons, there is an ongoing need and desire for improved systems and methods for facilitating self-administration of pills.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Implementations of the present disclosure extend at least to systems and methods for facilitating self-administration of pills.

Some embodiments provide a pill storage device that includes a pill storage casing. The pill storage device casing defines a compartment, and the compartment includes a compartment interior. The compartment interior includes a visual token. The visual token is detectable by a scanning sensor to indicate that contents have been removed from the compartment interior.

Some embodiments provide a system for facilitating self-administration of pills. The system includes a user device that includes a scanning sensor. The system also includes a pill storage device. The pill storage device includes a pill storage device casing defining a compartment. The compartment includes a compartment interior, and the compartment interior includes a visual token. The visual token is detectable by the scanning sensor. The system also includes one or more processors and one or more hardware storage devices storing instructions that are executable by the one or more processors to configure the system to perform various acts. The instructions are executable to configure the system to scan for the visual token with the scanning sensor of the user device. Detecting the visual token with the scanning sensor of the user device indicates that contents have been removed from the compartment interior of the pill storage device.

Some embodiments provide a method for facilitating self-administration of pills. The method includes activating an alarm in response to a triggering condition and scanning for a visual token of a pill storage device. The scanning is performed using a scanning sensor of a user device. The method also includes, in response to detecting the visual token with the scanning sensor of the user device, deactivating the alarm.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings.

FIG. 1 illustrates example components of a system for facilitating self-administration of pills;

FIG. 2A illustrates a front perspective view of a pill storage device in a partially open configuration;

FIG. 2B illustrates a top view of a pill storage device in an open configuration;

FIGS. 3A-3D illustrate an example system carrying out acts associated with facilitating self-administration of pills; and

FIG. 4 illustrates an example flow diagram depicting acts associated with facilitating self-administration of pills.

### DETAILED DESCRIPTION

Implementations of the present disclosure extend at least to systems and methods for facilitating self-administration of pills.

In some implementations, a pill storage device includes a pill storage casing. The pill storage device casing defines a compartment, and the compartment includes a compartment interior. The compartment interior includes a visual token. The visual token is detectable by a scanning sensor to indicate that contents have been removed from the compartment interior.

Some embodiments provide a system for facilitating self-administration of pills. The system includes a user device that includes a scanning sensor. The system also includes a pill storage device. The pill storage device includes a pill storage device casing defining a compartment. The compartment includes a compartment interior, and the compartment interior includes a visual token. The visual token is detectable by the scanning sensor. The system also includes one or more processors and one or more hardware storage devices storing instructions that are executable by the one or more processors to configure the system to perform various acts. The instructions are executable to configure the system to scan for the visual token with the scanning sensor of the user device. Detecting the visual token with the scanning sensor of the user device indicates that contents have been removed from the compartment interior of the pill storage device.

In at least some implementations of the present disclosure, a method for facilitating self-administration of pills includes activating an alarm in response to a triggering condition and scanning for a visual token of a pill storage device. The scanning is performed using a scanning sensor of a user device. The method also includes, in response to detecting the visual token with the scanning sensor of the user device, deactivating the alarm.

Those skilled in the art will recognize, in view of the present disclosure, that at least some of the disclosed embodiments may address various shortcomings associated with user self-administration of pills. For example, although conventional systems may utilize alarms to remind users to ingest one or more pills according to a schedule, users are often able to immediately silence such alarms whether or not they have obtained or ingested the one or more pills intended for ingestion in response to the alarm. Thus, users often forget to ingest their pills despite the use of an alarm to remind them to do so.

In contrast, in some implementations of the present disclosure, an alarm that reminds a user to ingest one or more pills is only deactivated by scanning a visual token associated with a compartment of a pill storage device that stores the one or more pills. The visual token may remain at least partially obscured while the one or more pills are positioned within the compartment. Removal of the one or more pills from the compartment may expose the visual token such that the visual token becomes detectable by a scanning device.

Thus, in some instances, implementations of the present disclosure may prevent users from silencing a pill administration alarm until after the user removes the pills from a pill repository. Accordingly, implementations of the present disclosure may improve the ability of users to adhere to pill self-administration schedules and may reduce the incidence of users forgetting to ingest pills in response to an alarm therefor.

In view of the foregoing, implementations of the present disclosure may increase the efficacy of medicinal treatment regimens and/or may improve patient outcomes. Furthermore, implementations of the present disclosure may allow users to ingest non-medicinal pills (e.g., vitamins, supplements, minerals, ingredients, and/or others) according to a desired schedule with increased accuracy.

One will appreciate, in view of the present disclosure, that a “pill” refers broadly to any type of ingestible product, whether medicinal or not and regardless of the form. For example, a pill may be implemented as a capsule, tablet, chewable, or other form.

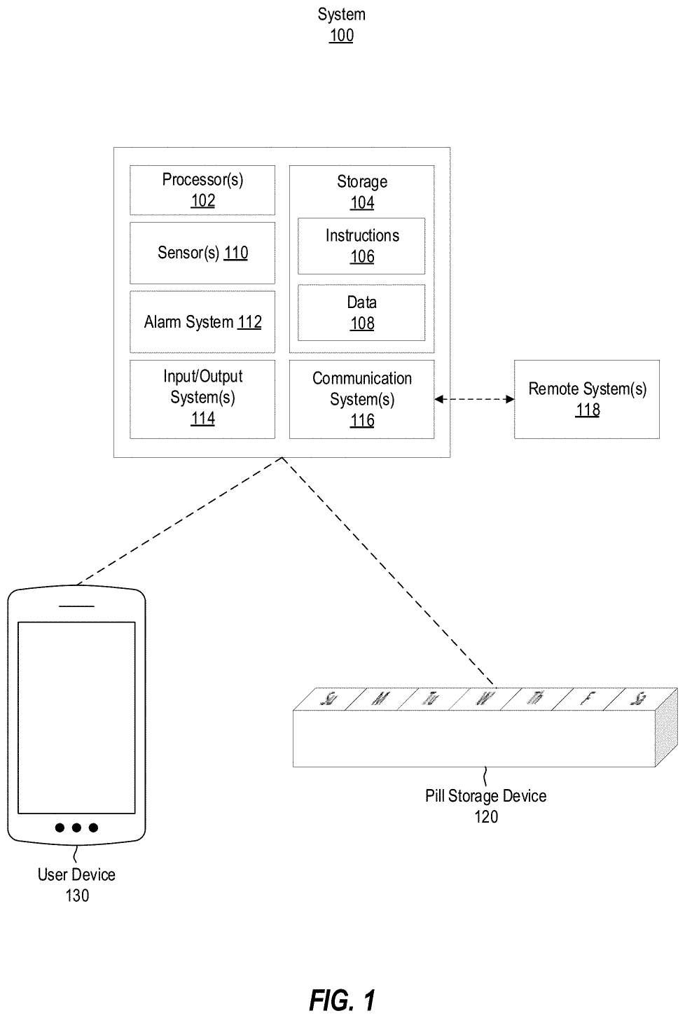

Having described some of the various high-level features and benefits of the disclosed embodiments, attention will now be directed to FIGS. 1 through 4. These Figures illustrate various supporting illustrations related to the disclosed embodiments.

FIG. 1 illustrates various example components of a system 100 for facilitating self-administration of pills, in accordance with the present disclosure. For example, FIG. 1 illustrates that a system 100 may include processor(s) 102, storage 104, sensor(s) 110, an alarm system 112, input/output system(s) 114 (I/O system(s) 114), and communication system(s) 116. Although FIG. 1 illustrates a system 100 as including particular components, one will appreciate, in view of the present disclosure, that a system 100 may comprise any number of additional or alternative components.

The processor(s) 102 may comprise one or more sets of electronic circuitry that include any number of logic units, registers, and/or control units to facilitate the execution of computer-readable instructions (e.g., instructions that form a computer program). Such computer-readable instructions may be stored within storage 104. The storage 104 may comprise physical system memory and may be volatile, non-volatile, or some combination thereof. Furthermore, storage 104 may comprise local storage, remote storage (e.g., accessible via communication system(s) 116 or otherwise), or some combination thereof. Additional details related to processors (e.g., processor(s) 102) and computer storage media (e.g., storage 104) will be provided hereinafter.

As will be described in more detail, the processor(s) 102 may be configured to execute instructions 106 stored within storage 104 to perform certain actions associated with facilitating self-administration of pills. The actions may rely at least in part on data 108 stored on storage 104 in a volatile or non-volatile manner.

In some instances, the actions may rely at least in part on communication system(s) 116 for receiving data and/or instructions from remote system(s) 118, which may include, for example, separate systems or computing devices, sensors, and/or others. The communications system(s) 118 may comprise any combination of software or hardware components that are operable to facilitate communication between on-system components/devices and/or with off-system components/devices. For example, the communications system(s) 118 may comprise ports, buses, or other physical connection apparatuses for communicating with other devices/components. Additionally, or alternatively, the communications system(s) 118 may comprise systems/components operable to communicate wirelessly with external systems and/or devices through any suitable communication channel(s), such as, by way of non-limiting example, Bluetooth, ultra-wideband, Wi-Fi, WLAN, infrared communication, and/or others.

FIG. 1 illustrates that a system 100 may comprise or be in communication with sensor(s) 110. Sensor(s) 110 may comprise any device for capturing or measuring data representative of perceivable phenomenon. By way of non-limiting example, the sensor(s) 110 may comprise one or more image sensors, optical scanners, microphones, thermometers, barometers, magnetometers, accelerometers, gyroscopes, and/or others. As will be described in more detail hereinafter, sensor(s) 110 may include any image sensor and/or scanning device usable to detect the presence of a visual token.

Furthermore, FIG. 1 illustrates that a system 100 may comprise or be in communication with I/O system(s) 114. I/O system(s) 114 may include any type of input or output device such as, by way of non-limiting example, a display, a touch screen, a mouse, a keyboard, a controller, a speaker, a light source, a vibration motor, and/or others, without limitation. In some instances, I/O system(s) 114 may at least partially rely on the sensor(s) 110 (e.g., via a microphone or motion sensor system to detect user input).

FIG. 1 illustrates that a system 100 may comprise an alarm system 112, which may be configurable to activate an alarm that is perceivable by humans. For example, activation of an alarm may include presentation of one or more audible, visible, tactile, or even smellable stimuli for perception by human users. In this regard, an alarm system 112 may comprise or utilize any components described herein (e.g., processor(s) 102, storage 104, communication system(s) 116, I/O system(s) 114 such as a speaker, a display, a light source, a vibration motor, etc.) and/or may comprise dedicated components for activating an alarm. As will be described in more detail hereinafter, an alarm may be used to cause a user to obtain one or more pills, and the alarm may be deactivated by scanning/detection of a visual token.

FIG. 1 conceptually represents that the components of the system 100 may be implemented on a pill storage device 120 and/or on a user device 130 under any configuration. For example, sensor(s) 110 for detecting a visual token may be implemented on the user device 130. Furthermore, processor(s) 102 and/or storage 104 for carrying out acts associated with facilitating self-administration of pills, as described herein, may be implemented on the user device 130, the pill storage device 120, or both (e.g., both the user device 130 and the pill storage device 120 may both comprise separate processors and/or storage). In some instances, a pill storage device 120 and a user device 130 both comprise communication system(s) 116 such that the pill storage device 120 and the user device 130 are configured to communicate with one another (e.g., the pill storage device 120 may receive signals from the user device to carry out actions). In this regard, any processing associated with facilitating self-administration of pills, in accordance with the present disclosure, may be performed by the pill storage device 120, the user device, the remote system(s) 118, or combinations thereof.

As another example, an alarm system 112 may be implemented on the pill storage device 120, the user device 130, or both (e.g., where both the user device 130 and the pill storage device 120 comprise separate alarm systems, or where both the user device 130 and the pill storage device 120 operate in cooperation to facilitate alarm system functionality).

FIG. 1 conceptually represents the user device 130 as a mobile electronic device (e.g., a smartphone or a tablet). However, one will appreciate, in view of the present disclosure, that a user device 130 may take on any suitable form. FIG. 1 conceptually represents the pill storage device 120 as a device capable of storing one or more pills (e.g., separate pills for each day of a week, as visually depicted on the pill storage device 120 in FIG. 1 via the characters “Su”, “M”, “Tu”, “W”, “Th”, “F”, and “Sa” disposed on the pill storage device 120 and representing, respectively, Sunday, Monday, Tuesday, Wednesday, Thursday, Friday, and Saturday). Additional details related to the pill storage device 120 will be provided hereinafter.

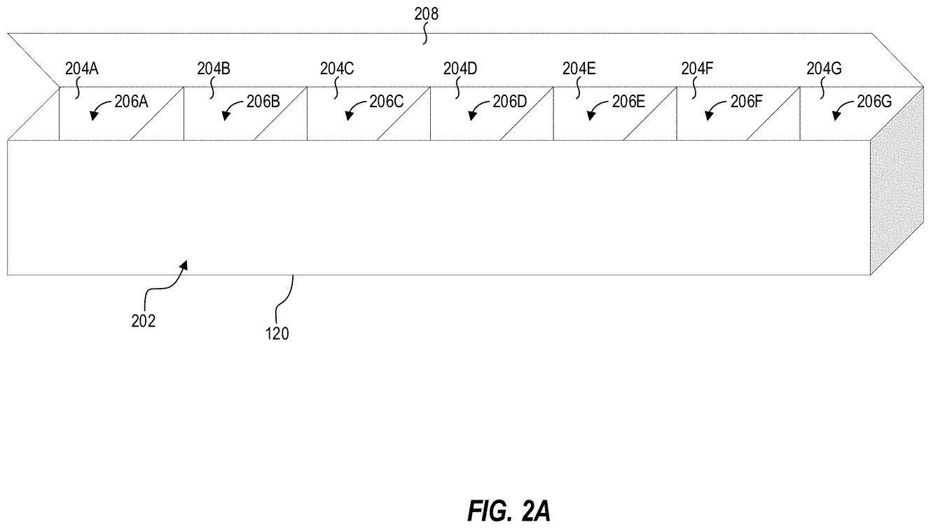

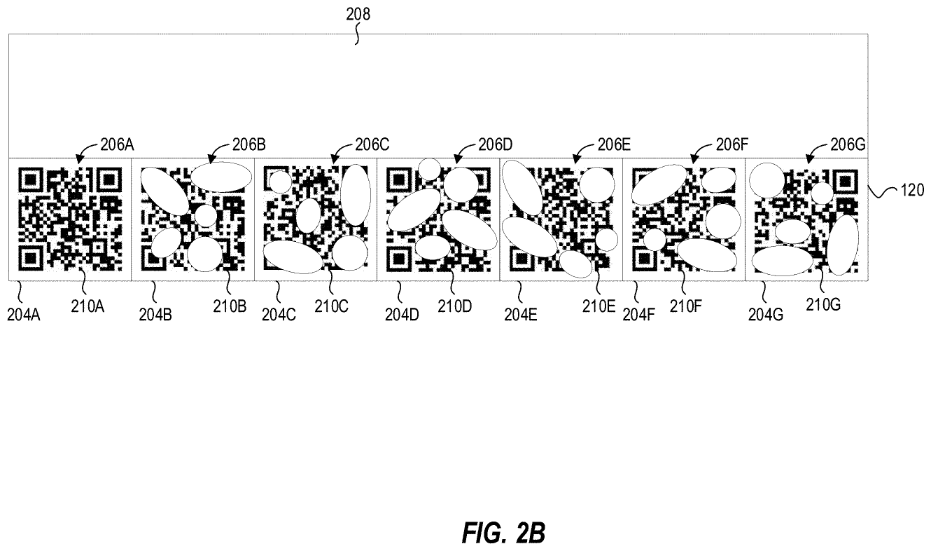

FIG. 2A illustrates a front perspective view of a pill storage device 120 in a partially open configuration. As noted above, the pill storage device 120 is configured for the storage of one or more pills. In this regard, the pill storage device 120 includes a pill storage device casing 202 that defines a plurality of compartments 204A, 204B, 204C, 204D, 204E, 204F, and 204G. The pill storage device casing 202 may comprise any suitable material (e.g., a polymer material). In the example shown in FIG. 2A, each separate compartment of the plurality of compartments 204A-204G are associated with a separate corresponding day of a week (e.g., Sunday, Monday, Tuesday, Wednesday, Thursday, Friday, and Saturday, respectively).

As shown in FIG. 2A, each of the plurality of compartments 204A-204G of the pill storage device 120 includes a respective compartment interior 206A, 206B, 206C, 206D, 206E, 206F, and 206G. The compartment interiors 206A-206G of the pill storage device 120 of FIG. 2A are sized and shaped to receive and store one or more pills. In this way, a user may place various pills within the various compartments 204A-204G of the pill storage device 120 for ingestion by the user according to a pill administration schedule (e.g., to self-administer separate sets of pills on separate days).

The pill storage device 120 of FIG. 2A includes a cover 208 that is selectively openable to expose the compartment interiors 206A-206G. Although FIG. 2A shows a single cover 208 for exposing all of the compartment interiors 206A-206G, one will appreciate, in view of the present disclosure, that the compartment interiors 206A-206G may be exposable by any number of covers (e.g., a separate cover for covering each compartment interior 206A-206G).

Although FIG. 2A illustrates a pill storage device 120 that includes a particular number of compartments and compartment interiors, a pill storage device 120 may comprise any number of compartments and/or compartment interiors in accordance with the present disclosure (e.g., one or more compartments). For example, a pill storage device 120 may comprise only a single compartment or may comprise two separate compartments for each day of a week (e.g., an “a.m.” compartment and a “p.m.” compartment), resulting 14 compartments. In some instances, the number of compartments available in a pill storage device 120 is selectively modifiable by a user, such as where the pill storage device 120 is configured to receive partitions for defining compartment interiors.

FIG. 2B illustrates a top view of a pill storage device 120 with the cover 208 thereof in an open configuration, exposing the compartments 204A-204G and respective compartment interiors 206A-206G thereof. As is evident from FIG. 2B, the pill storage device 120 includes a visual token 210A, which, as will be described in more detail hereinafter, is scannable or detectable by a scanning sensor (e.g., sensor(s) 110 of a user device 130). The visual token 210A is positioned within the compartment interior 206A of compartment 204A. FIG. 2B shows the visual token 210A as being sized and positioned within the compartment interior 206A such that the visual token 210A is arranged to become at least partially obscured from view (e.g., relative to a vantage point outside of the opening of the compartment 204A) when one or more pills are positioned within the compartment interior 206A.

In particular, FIG. 2B shows the visual token 210A located on a bottom or floor surface of the compartment interior 206A. Thus, when a pill becomes positioned within the compartment 204A and over the visual token 210A, the pill operates to cover a portion of the visual token 210A. For example, FIG. 2B also illustrates visual tokens 210B, 210C, 210D, 210E, 210F, and 210G positioned, respectively, within compartment interiors 206B, 206C, 206D, 206E, 206F, and 206G. Each of the compartment interiors 206B-206G includes five pills, and FIG. 2B shows that the pills operate to at least partially obscure the visual tokens 210B-210G.

In this way, the presence of one or more pills within a compartment interior 206A-206G may cause a scanning sensor attempting to detect or scan a visual token 210A-210G to fail to detect a visual token 210A-210G. For instance, a scanning sensor attempting to detect or scan one of the visual tokens 210B-210G would fail to detect the visual tokens 210B-210G in view of the pills positioned within the compartment interiors 206B-206G. In contrast, the absence of one or more pills within a compartment interior 206A-206G may enable a scanning sensor attempting to detect or scan a visual token 210A-210G to succeed in detecting a visual token 210A-210G. For instance, a scanning sensor attempting to detect or scan the visual token 210A would succeed in detecting the visual token 210A in view of the absence of pills positioned within the compartment interior 206A.

In this regard, a scanning sensor successfully detecting a visual token (e.g., one or more of visual tokens 210A-210G) may indicate that contents (e.g., pills, capsules, etc.) have been removed from a corresponding compartment interior (e.g., one or more of compartment interiors 206A-206G). In contrast, a scanning sensor failing to detect a visual token (e.g., one or more of visual tokens 210A-210G) may indicate that contents (e.g., pills) have not been removed from a corresponding compartment interior (e.g., one or more of compartment interiors 206A-206G). As will be described in more detail hereinafter, a detection of a visual token by a scanning sensor may be used to deactivate an alarm for reminding a user to self-administer one or more pills and thereby improve the likelihood that the alarm will be effective in causing the user to self-administer the one or more pills.

Although FIG. 2B represents the visual tokens 210A-210G as a quick response code (QR code), a visual token may take on any suitable form that is detectable by an optical sensor and/or image sensor. For instance, a visual token may comprise one or more barcodes, symbols, characters, colors, graphics, designs, structural features/elements, combinations thereof, and/or others. By way of additional example, a visual token may comprise the visual appearance of one or more empty compartment interiors of one or more compartments of a pill storage device. For instance, an optical sensor and/or image sensor may sense an image of a compartment interior (or an image of multiple compartment interiors), and a computing system may be configured (e.g., via artificial intelligence) to determine whether the compartment interior(s) represented in the image is/are empty or include(s) pills positioned therein.

FIG. 2B illustrates each separate visual token 210A-210G as being unique relative to the other visual tokens 210A-210G. For instance, visual token 210A comprises a QR code that is different from the QR codes depicted as visual tokens 210B-210G. In some instances, however, two or more compartment interiors 206A-206G may comprise corresponding visual tokens 210A-210G that are substantially identical to one another (e.g., two or more visual tokens may be represented by the same QR code). Furthermore, different compartment interiors may be associated with different types of visual tokens (e.g., where one compartment interior comprises a QR code, whereas another compartment interior comprises a symbol).

Furthermore, although FIG. 2B shows the visual tokens 210A-210G positioned on the bottom surfaces of the respective compartment interiors 206A-206G, one will appreciate, in view of the present disclosure, that a visual token may be positioned on any surface or combination of surfaces (whether associated with a compartment interior or not).

In addition, in some implementations where the compartments 204A-204G of the pill storage device 120 are selectively configurable/reconfigurable, the visual tokens 210A-210G may also be reconfigurable. For example, visual tokens may be provided with a pill storage device 120 in the form of reconfigurable tiles that a user may use in combination with compartment partitions to form a desired pill storage configuration for a pill storage device 120.

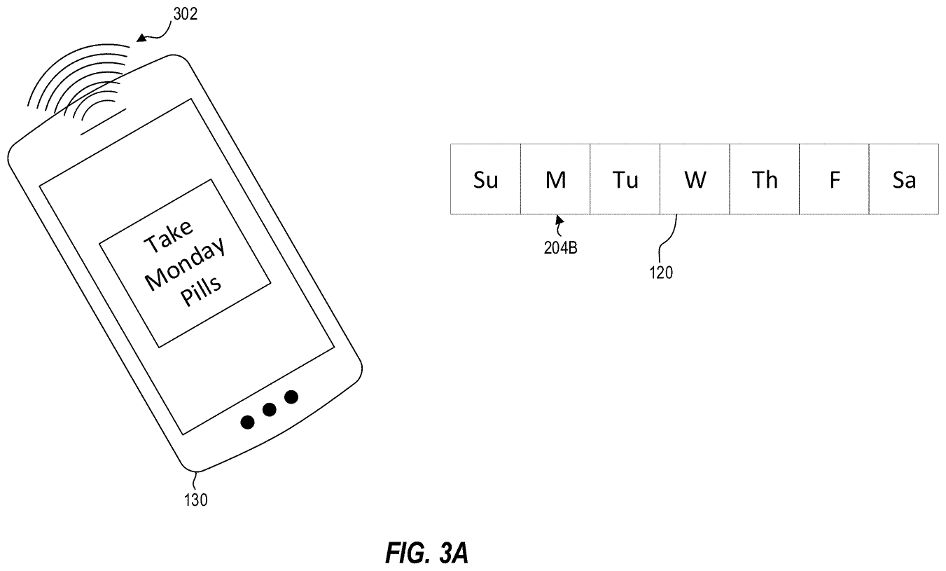

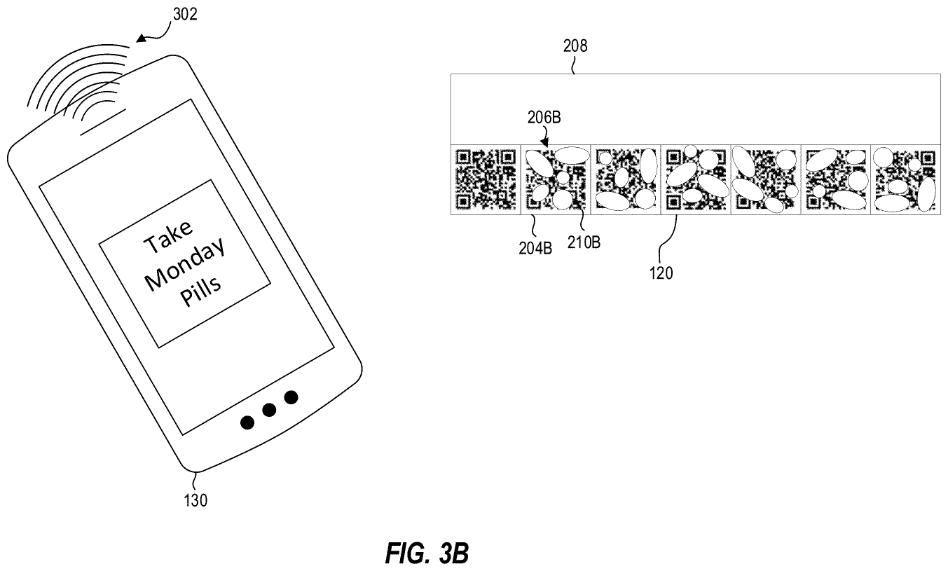

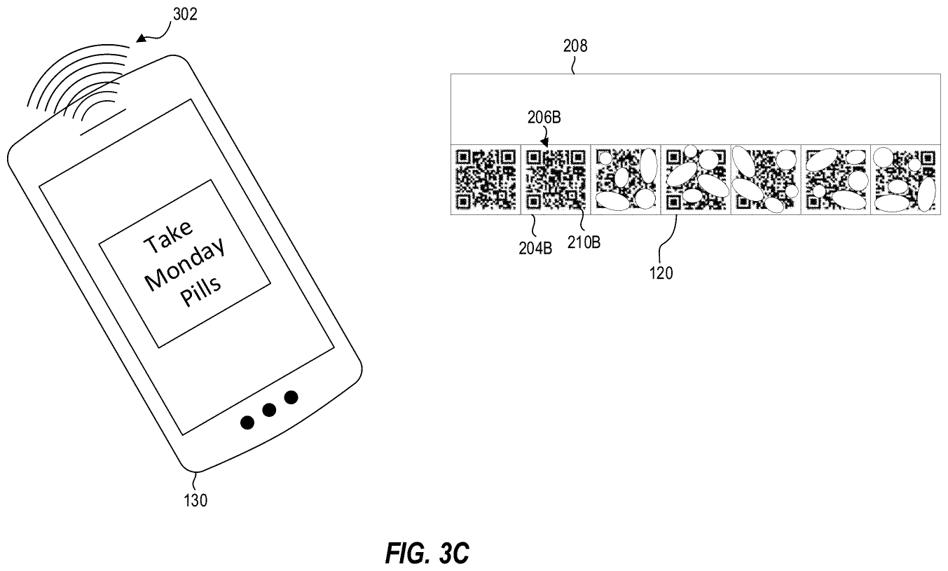

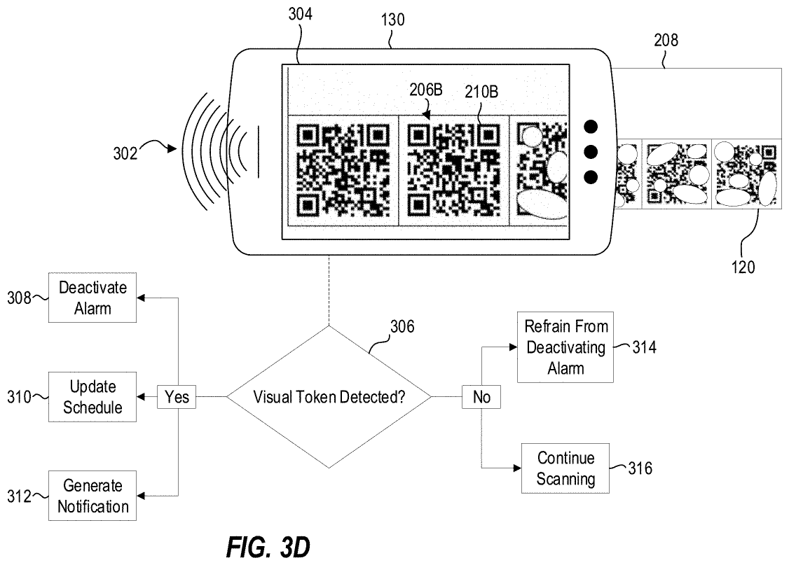

FIGS. 3A-3D illustrate an example system (e.g., corresponding to system 100) carrying out acts associated with facilitating self-administration of pills. In particular, FIG. 3A illustrates a system including a user device 130 and a pill storage device 120, as described hereinabove. As noted above, the user device 130 and/or the pill storage device 120 may comprise processor(s) 102, storage 104, an alarm system 112, and/or other components for executing instructions associated with facilitating self-administration of pills.

Accordingly, FIG. 3A shows the user device 130 activating an alarm 302 (e.g., in accordance with instructions 106). The alarm 302 may be activated in response to a triggering condition, such as detecting the presence of a target time. In one example, a user may predefine a pill administration schedule (e.g., via user input at the user device 130 or another system), which may define times and/or dates at which a user should self-administer pills. A pill administration schedule may also define the types of pills that a user should ingest (e.g., to assist users in loading compartments of a pill storage device 120 with pills). One will appreciate, in view of the present disclosure, that a user who defines the pill administration schedule need not be the same user that self-administers pills.

In some instances, the alarm 302 may be activated in response to detecting that a predetermined time period has elapsed. For example, some medicines achieve optimal results when precise temporal offsets are implemented between administration of doses. Accordingly, an alarm 302 may be triggered in response to determining that a predetermined time period has elapsed since administration of a preceding dose (which may correspond to a predetermined time period after deactivation of an alarm, as described hereinbelow).

The alarm 302 is visually represented in FIG. 3A by sound waves emitting from the user device 130, but, as noted above, an alarm may comprise additional or alternative presentations for perception by humans (e.g., light pulsing, image/graphic display, vibrating, etc.). In some instances, the alarm 302 functions to direct users to obtain one or more pills from the pill storage device 120 and self-administer the pills in response to the alarm 302. For instance, FIG. 3A shows a display of the user device 130 presenting the text, “Take Monday Pills” for viewing by users, which may direct users to obtain and ingest one or more pills from the compartment 204B of the pill storage device 120 associated with “Monday”. Accordingly, the alarm 302 may be configured to communicate which pill(s) are intended for current ingestion by a user, in addition to reminding the user to ingest the pill(s).

As noted above, the alarm 302 may be effectuated by an alarm system 112 that may comprise dedicated software and/or hardware components and/or that may at least partially leverage alarm functionality native to an existing device (e.g., existing alarm software and/or hardware associated with the user device 130 or another device). Furthermore, as noted hereinabove, the pill storage device may be configured to output an alarm that is additional or alternative to the alarm 302 depicted in FIG. 3A.

FIG. 3B illustrates the pill storage device 120 with the cover 208 thereof in an open position. For example, a user who perceived the alarm 302 may have become motivated to begin self-administering the pills associated with compartment 204B (e.g., the “Monday” pills) and therefore opened the cover 208 of the pill storage device 120, thereby exposing the compartment interior 206B of the compartment 204B. As noted above, the compartment interior 206B includes a visual token 210B, which is at least partially obscured by pills positioned within the compartment interior 206B. The user's motivation to begin self-administration of pills in response to the alarm 302 may be strengthened where the alarm 302 is primarily deactivated by scanning the visual token 210B that becomes scannable after removal of the pills of the compartment interior 206B (e.g., the “Monday” pills).

FIG. 3C shows the pill storage device 120 after the pills previously positioned within the compartment interior 206B of compartment 204B have been removed. For example, continuing with self-administration of pills responsive to the alarm 302 (and, in some instances, in order to deactivate the alarm 302), a user may proceed after opening the cover 208 of the pill storage device 120 to remove and ingest the “Monday” pills from the compartment interior 206B of compartment 204B.

As is evident from FIG. 3C, removing the pills from the compartment interior 206B provides an unobstructed view of the visual token 210B positioned within the compartment interior 206B (e.g., relative to a vantage point outside of the compartment interior 206B). In some instances, therefore, removing the pills from the compartment interior 206B renders the visual token 210B detectable by a scanning sensor, whereas the visual token 210B would remain undetectable by a scanning sensor were the pills to remain within the compartment interior 206B.

Accordingly, systems and methods of the present disclosure may ensure continuance of the alarm 302 until pills have been removed from an appropriate repository to expose a corresponding visual token (allowing, in some implementations, deactivation of the alarm 302 by scanning), which can increase user success in adhering to a pill self-administration schedule (e.g., by reducing the incidence of users deactivating a pill administration alarm without first obtaining one or more designated pills and thereafter neglecting to ingest the one or more designated pills).

FIG. 3D illustrates the user device 130 scanning (or being operated to scan) for the visual token 210B that has been exposed by removal of the pills from the compartment interior 206B of the pill storage device 120. As indicated hereinabove, a user device 130 may comprise sensor(s) 110, such as optical scanners and/or image sensors that may facilitate detection of the visual token 210B. In some instances, the sensor(s) 110 for detecting a visual token may at least partially leverage image sensing functionality native to a user device (e.g., exiting camera and/or image detection functionality of a user device 130).

The user device 130 may initiate scanning for a corresponding visual token (e.g., visual token 210B for an alarm 302 directing a user to ingest pills associated with compartment interior 206B) in response to various inputs and/or conditions. For example, in some instances, the user device 130 automatically initiates scanning for a corresponding visual token in response to detecting the triggering condition that causes activation of the alarm 302 (or in response to activation of the alarm itself). In some implementations, the user device 130 is configured to automatically begin scanning after a predetermined time period lapses following the detection of a triggering condition or event. In some instances, the user device 130 initiates scanning for a corresponding visual token in response to user input interacting with the user device 130 and/or the pill storage device. For example, a user may provide input on a touch screen or button of the user device 130, and the user device 130 may begin scanning for a corresponding visual token in response to such input. As yet another example, a user may provide input at the pill storage device (e.g., by pressing a button or triggering a sensor, such as by opening the cover 208 of the pill storage device 120 to trigger a sensor for detecting opening of the pill storage device), and the user device may begin scanning for a corresponding visual token in response to such input.

FIG. 3D conceptually represents the scanning by the user device 130 for the visual token 210B by the user device 130 displaying (on a display thereof) a representation of an image 304 captured using the scanning sensor(s) of the user device 130. The image 304 includes a representation of the visual token 210B, the compartment interior 206B, and other elements. One will appreciate, in view of the present disclosure, that a user device (or other system) may comprise any hardware or software components (including computer-executable instructions) necessary to determine whether a captured image (or other sensor data) includes a representation of a visual token (e.g., QR code detection components, barcode detection components, object/feature/character/symbol recognition components, etc.).

As indicated hereinabove, the detection of a visual token corresponding to an alarm (e.g., visual token 210B of compartment interior 206B, which corresponds to “Monday” and therefore corresponds to the alarm 302 directing a user to ingest the “Monday” pills) indicates that one or more pills (or other contents) have been displaced to make the visual token detectable by a scanning sensor, thereby indicating that a user has at least handled the one or more pills and thereby increasing the likelihood that the user has already ingested or will soon ingest the one or more pills in response to the alarm (e.g., and in accordance with a pill administration schedule). Thus, the detection of a visual token corresponding to an alarm may be operable to trigger additional action for facilitating self-administration of pills, in accordance with the present disclosure.

FIG. 3D focuses on an example of an alarm 302 being activated for causing a user to access the pills of compartment interior 206B and a scanning sensor of a user device 130 scanning for a visual token 210B that corresponds to the alarm 302 (e.g., also corresponding to the compartment 204B) to determine whether the user obtained the pills from compartment interior 206B. A successful detection of the visual token 210B indicates that the user obtained the pills from compartment 204B. One will appreciate, in view of the present disclosure, that different alarms associated with causing users to access different corresponding pills (e.g., pills of compartment interiors 206A, 206C, 206D, 206E, 206F, 206G, etc.) may cause a user device 130 to scan for different corresponding visual tokens (e.g., visual tokens 210A, 210C, 210D, 210E, 210F, 210G, etc.) and determine whether the user obtained the different corresponding pills based on successful detection of the different corresponding visual tokens.

It should be noted that, in some instances, a system is configured to determine whether a user successfully obtained appropriate pills based on detection of multiple visual tokens. For instance, as noted above, FIG. 3D focuses on an example in which each separate compartment interior is associated with a unique visual token relative to the other compartment interiors (e.g., distinct QR codes for each compartment interior). However, in other instances, each separate compartment interior includes a visual token that is substantially similar to the visual tokens of other compartment interiors (e.g., the same QR code for each compartment interior, the visual appearance of an empty compartment interior that is substantially the same for each compartment interior, etc.). In such other instances, in some implementations, the system may determine whether a user successfully obtained appropriate pills based on the number of substantially similar visual tokens detected (e.g., the number of QR codes detected, the number of compartment interiors with an empty visual appearance, etc.).

For example, where an alarm directs a user to obtain and ingest pills associated with a third compartment interior of a pill storage device (when pills associated with a first compartment interior and a second compartment interior of the pill storage device have previously been removed), a system may determine that the user has obtained the corresponding pills of the third compartment interior based on detecting, with a scanning sensor, three instances of a substantially identical visual token (e.g., by detecting a visual token from each of the first three compartment interiors of the pill storage device).

In this regard, although FIG. 3D focuses on a scanning sensor only capturing a particular portion of the pill storage device 120 to determine whether appropriate pills were removed therefrom, a scanning sensor may capture any portion (e.g., a complete top view) of a pill storage device to determine whether appropriate pills were removed therefrom.

As indicated above, the detection of a visual token corresponding to an alarm, or the failure to detect such a visual token, may be operable to trigger additional action. FIG. 3D provides conceptual representations of such additional action. For example, FIG. 3D shows a decision block 306 connected via a dashed line to the user device 130 scanning for the visual token 210B. The decision block 306 includes the text “Visual Token Detected?” indicating that the system may be configured to determine, based on sensor data obtained by a scanning sensor, whether a particular visual token (or combination of visual tokens) has been detected.

In response to determining that a particular visual token has been detected by a scanning sensor of the user device 130 (indicated by the “Yes” block connected to the left side of the decision block 306), a system may deactivate the alarm 302 (e.g., represented in FIG. 3D by block 308). By primarily allowing deactivation of the alarm 302 by successfully scanning of a visual token, systems and methods of the present disclosure may improve user self-administration of pills by advantageously preventing users from deactivating an alarm until after displacing pills intended for ingestion.

Successful detection of a visual token may additionally or alternatively trigger other actions. For instance, a system may be configured to update a pill administration schedule or pill administration tracking data structure in response to detecting a visual token (and thereby inferring successful pill administration), as represented in FIG. 3D by block 310. By tracking successful pill administrations, users may ensure adherence to a pill administration schedule and may even accurately track pill administration times to draw additional correlations between treatment effectiveness and pill administration timing.

In some implementations, a system is configured to generate a notification in response to detecting a visual token (and thereby inferring successful pill administration), as represented in FIG. 3D by block 312. The notification may be automatically communicated to various entities, such as systems associated with one or more individuals interested in whether a user successfully ingested pills according to a pill administration schedule (e.g., medical practitioners, caregivers, guardians, relatives, etc.).

In response to determining that a particular visual token has not been detected by a scanning sensor of the user device 130 (indicated by the “No” block connected to the right side of the decision block 306), a system may refrain from deactivating the alarm 302 (e.g., represented in FIG. 3D by block 314). A system may interpret failure to detect a visual token as an indication that one or more pills intended for present ingestion have not yet been removed from the pill storage device 120. In this way, the alarm 302 may continue to be presented for perception by users in order to continue to motivate users to obtain one or more pills from the compartment interior 206B and scan the visual token 210B to deactivate the alarm 302.

Furthermore, in response to determining that a particular visual token has not been detected by a scanning sensor of the user device 130, a system may continue scanning for the particular visual token, as represented in FIG. 3D by block 316. For instance, the user device 130 may persist in a state of scanning for the visual token 210B (e.g., continue detecting images with the sensor(s) 110 of the user device 130) until the user device 130 successfully detects the visual token 210B.

Although the present disclosure focuses, in at least some respects, on self-administration of pills (whether medicinal or not), it should be noted that implementations of the present disclosure extend to any exhaustible product for use or consumption according to a schedule, such as, by way of non-limiting example, effervescent products, powder products, injectable products, beverage products, electrical or mechanical devices (e.g., for maintenance purposes), and/or others.

Furthermore, in accordance with the present disclosure, a visual token need not be confined to a compartment and a product intended for use in response to an alarm need not be intended for complete exhaustion in response to the alarm. By way of illustrative example, an alarm may be used to remind a user to use a multiple-use product (e.g., a bottle of lotion, a toothbrush, etc.), and the multiple-use product may be placed on a surface (e.g., a table or counter) at least partially obscuring a visual token positioned on the surface. In this way, the alarm may cause the user to obtain the multiple-use product in a manner that exposes the visual token, allowing the user to scan the visual token to silence the alarm.

The following discussion now refers to a number of methods and method acts that may be performed by the disclosed systems. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

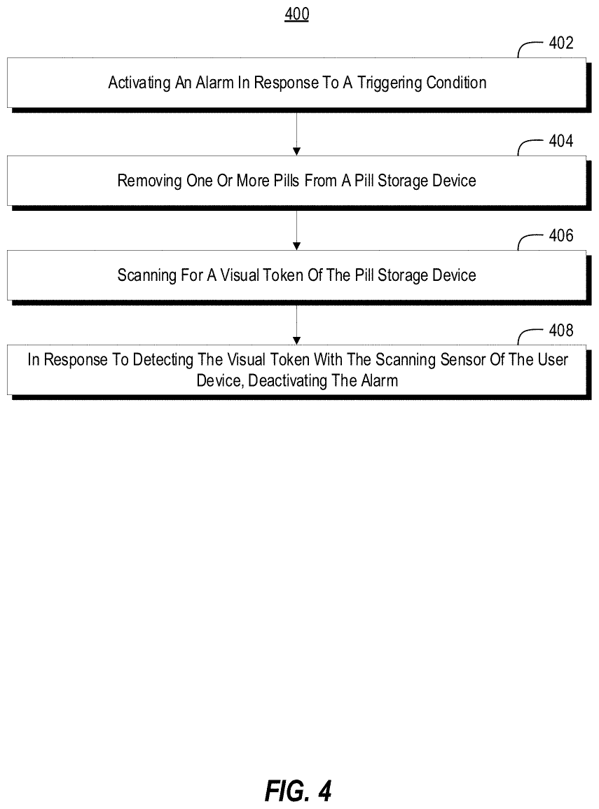

FIG. 4 illustrates an example flow diagram 400 depicting acts associated with facilitating self-administration of pills. The discussion of the various acts represented in the flow diagram 400 includes reference to various hardware components described in more detail with reference to FIGS. 1-3D.

Act 402 of flow diagram 400 includes activating an alarm in response to a triggering condition. Act 402 is performed, in some instances, by a system 100 utilizing processor(s) 102, storage 104, sensor(s) 110, an alarm system 112, input/output system(s) 114, communication system(s) 116, and/or other components, which may be implemented on a user device 130, a pill storage device 120, a combination thereof, and/or another device. In some implementations, the triggering condition operable to cause activation of the alarm includes detecting that a predetermined time period has elapsed (e.g., a time period following a timepoint associated with a previous administration of one or more pills). In some instances, the triggering condition operable to cause activation of the alarm includes detecting the presence of a target time (e.g., a pill administration time in accordance with a pill administration schedule).

Act 404 of flow diagram 400 includes removing one or more pills from a pill storage device. Act 404 is performed, in some instances, by a user associated with a user device 130 and/or a pill storage device 120. In some implementations, removing the one or more pills from the pill storage device exposes a visual token disposed. In some embodiments, the visual token is sized and positioned within a compartment interior of the pill storage device to be at least partially obscured when one or more pills are positioned within the compartment interior. In this regard, detecting the visual token with the scanning sensor of a user device may indicate that the one or more pills have been removed from the compartment interior of the pill storage device.

Act 406 of flow diagram 400 includes scanning for a visual token of the pill storage device. Act 406 is performed, in some instances, by a system 100 utilizing processor(s) 102, storage 104, sensor(s) 110, input/output system(s) 114, communication system(s) 116, and/or other components, which may be implemented on a user device 130, a pill storage device 120, a combination thereof, and/or another device. The scanning may be performed at least in part using a scanning sensor of a user device 130. In some implementations, the user device automatically initiates scanning for the visual token with the scanning sensor thereof in response to the triggering condition for activating the alarm or in response to activation of the alarm (discussed above with reference to act 402). As noted above, detecting the visual token with the scanning sensor of the user device may indicate that contents have been removed from the compartment interior of the pill storage device.

Act 408 of flow diagram 400 includes, in response to detecting the visual token with the scanning sensor of the user device, deactivating the alarm. Act 408 is performed, in some instances, by a system 100 utilizing processor(s) 102, storage 104, sensor(s) 110, an alarm system 112, input/output system(s) 114, communication system(s) 116, and/or other components, which may be implemented on a user device 130, a pill storage device 120, a combination thereof, and/or another device. Such functionality may, in some instances, improve upon alarm systems for causing users to self-administer pills by preventing users from deactivating a pill administration alarm without first obtaining the pill(s) intended for present ingestion.

A system may perform additional or alternative acts in response to detecting the visual token with the scanning sensor of the user device, such as, by way of non-limiting example, updating a pill administration schedule or pill administration tracking data structure, and/or generating a notification indicating that the visual token was successfully detected and thereby inferring successful self-administration of pills.

Furthermore, a system may perform various acts in response to failing to detect the visual token with the scanning sensor of the user device, such as refraining from deactivating the alarm (inferring failure to self-administer pills) and/or continuing to scan for the visual token with the scanning sensor of the user device.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

In some implementations, systems of the present disclosure may comprise or be configurable to execute any combination of software and/or hardware components that are operable to facilitate processing using machine learning models or other artificial intelligence-based structures/architectures. For example, one or more processors may comprise and/or utilize hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A pill storage device for facilitating self-administration of pills, comprising:
a pill storage device casing defining a compartment, the compartment comprising a compartment interior, wherein the compartment interior comprises a visual token, the visual token being detectable by a scanning sensor to indicate that contents have been removed from the compartment interior.

2. The pill storage device of claim 1, wherein the compartment is sized and shaped to receive one or more pills.

3. The pill storage device of claim 1, wherein the pill storage device comprises a cover that is selectively openable to expose the compartment interior.

4. The pill storage device of claim 1, wherein the visual token is sized and positioned within the compartment interior to be at least partially obscured when one or more pills are positioned within the compartment interior, such that a scanning sensor attempting to detect the visual token when the one or more pills are positioned within the compartment fails to detect the visual token.

5. The pill storage device of claim 4, wherein the visual token is positioned on a bottom surface of the compartment interior.

6. The pill storage device of claim 1, wherein the visual token comprises a scannable code.

7. The pill storage device of claim 1, wherein the pill storage device casing defines a plurality of compartments, each of the plurality of compartments comprising a respective compartment interior, and each respective compartment interior comprising a respective visual token.

8. The pill storage device of claim 7, wherein the respective visual token of at least one respective compartment interior is substantially identical to the respective visual token of at least another respective compartment interior.

9. A system for facilitating self-administration of pills, comprising:
a user device comprising a scanning sensor;
a pill storage device, comprising:
a pill storage device casing defining a compartment, the compartment comprising a compartment interior, wherein the compartment interior comprises a visual token, the visual token being detectable by the scanning sensor;
one or more processors; and
one or more hardware storage devices storing instructions that are executable by the one or more processors to configure the system to:
scan for the visual token with the scanning sensor of the user device, wherein detecting the visual token with the scanning sensor of the user device indicates that contents have been removed from the compartment interior of the pill storage device.

10. The system of claim 9, wherein the system further comprises an alarm system configured to activate an alarm, and wherein the instructions are executable by the one or more processors to further configure the system to:
activate the alarm of the alarm system in response to a triggering condition.

11. The system of claim 10, wherein the triggering condition comprises detecting that a predetermined time period has elapsed or detecting a presence of a target time.

12. The system of claim 10, wherein the instructions are executable by the one or more processors to further configure the system to:
automatically initiate scanning for the visual token with the scanning sensor of the user device in response to the triggering condition or in response to activation of the alarm.

13. The system of claim 10, wherein the instructions are executable by the one or more processors to further configure the system to:
deactivate the alarm of the alarm system in response to detecting the visual token with the scanning sensor of the user device.

14. The system of claim 10, wherein the instructions are executable by the one or more processors to further configure the system to:
in response to failing to detect the visual token with the scanning sensor of the user device, refrain from deactivating the alarm.

15. The system of claim 14, wherein the instructions are executable by the one or more processors to further configure the system to:
in response to failing to detect the visual token with the scanning sensor of the user device, continue to scan for the visual token with the scanning sensor of the user device.

16. The system of claim 10, wherein at least a portion of the alarm system is implemented on the pill storage device.

17. The system of claim 9, wherein at least some of the one or more processors or the one or more hardware storage devices are implemented on the pill storage device.

18. A method for facilitating self-administration of pills, comprising:
activating an alarm in response to a triggering condition;
scanning for a visual token of a pill storage device, the scanning being performed using a scanning sensor of a user device; and
in response to detecting the visual token with the scanning sensor of the user device, deactivating the alarm.

19. The method of claim 18, wherein the visual token is sized and positioned within a compartment interior of the pill storage device to be at least partially obscured when one or more pills are positioned within the compartment interior, such that detecting the visual token with the scanning sensor of the user device indicates that the one or more pills have been removed from the compartment interior of the pill storage device.

20. The method of claim 18, wherein the triggering condition comprises detecting that a predetermined time period has elapsed or detecting a presence of a target time.
